(19) United States
(12) Plant Patent Application Publication
(10) Pub. No.: US 2015/0305219 P1
(43) Pub. Date: Oct. 22, 2015

(54) PETUNIA PLANT NAMED 'KLEPH13228'

(50) ;

(71) Applicant: Klemm+Sohn GmbH & Co. KG, Stuttgart (DE)

(72) Inventors: Nils Klemm, Stuttgart (DE); Antonella Capo, Latina (IT)

(73) Assignee: Klemm+Sohn GmbH & Co. KG, Stuttgart (DE)

(21) Appl. No.: 14/120,065

(22) Filed: Apr. 21, 2014

(51) Int. Cl. A01H 5/00

(52) U.S. Cl. PLT/356.16

(57) ABSTRACT

A new petunia plant particularly distinguished by having single, light blue colored flowers, and an upright mounding, medium-compact vigour plant habit, is disclosed

### GENUS AND SPECIES

Petunia hybrida

### VARIETY DENOMINATION

‘KLEPH13228’

### BACKGROUND OF THE NEW PLANT

The present invention comprises a new and distinct variety of petunia, botanically known as Petunia hybrida and hereinafter referred to by the variety name ‘KLEPH13228’. ‘KLEPH13228’ is a selection from a cross-pollination of the proprietary female petunia line ‘PH-2008-0221’ (unpatented) and the proprietary male petunia line ‘PH-2008-0448’ (unpatented).

In July of 2009 the proprietary female petunia line ‘PH-2008-0221’ and the proprietary male petunia line ‘PH-2008-0448’ were cross-pollinated in Latina, Italy and seeds were obtained. The seeds were sown and plants were grown for evaluation. A plant line was selected in April 2010 in Latina, Italy and named ‘KLEPH13228’. In April 2010, ‘KLEPH13228’ was first vegetatively propagated by terminal tip cuttings and tissue culture. ‘KLEPH13228’ was found to reproduce true to type in successive generations of asexual propagation via terminal tip cuttings and tissue culture in Stuttgart, Germany.

### SUMMARY

The following are the most outstanding and distinguishing characteristics of this new variety when grown under normal horticultural practices in Stuttgart, Germany and Latina, Italy.

1. Single, light blue colored flowers; and

2. An upright, mounding, medium-compact vigour plant habit.

### DESCRIPTION OF THE PHOTOGRAPHS

This new petunia plant is illustrated by the accompanying photographs which show an overall view of the plant. The colors shown are as true as can be reasonably obtained by conventional photographic procedures.

FIG. 1 shows the overall plant habit of the plant grown in a basket. The plant is about 5 months-old grown in Stuttgart, Germany in July 2013.

FIG. 2 shows a close-up of the mature inflorescence of the plant grown in a pot. The plant is about 3 months-old grown in Stuttgart, Germany in May 2013.

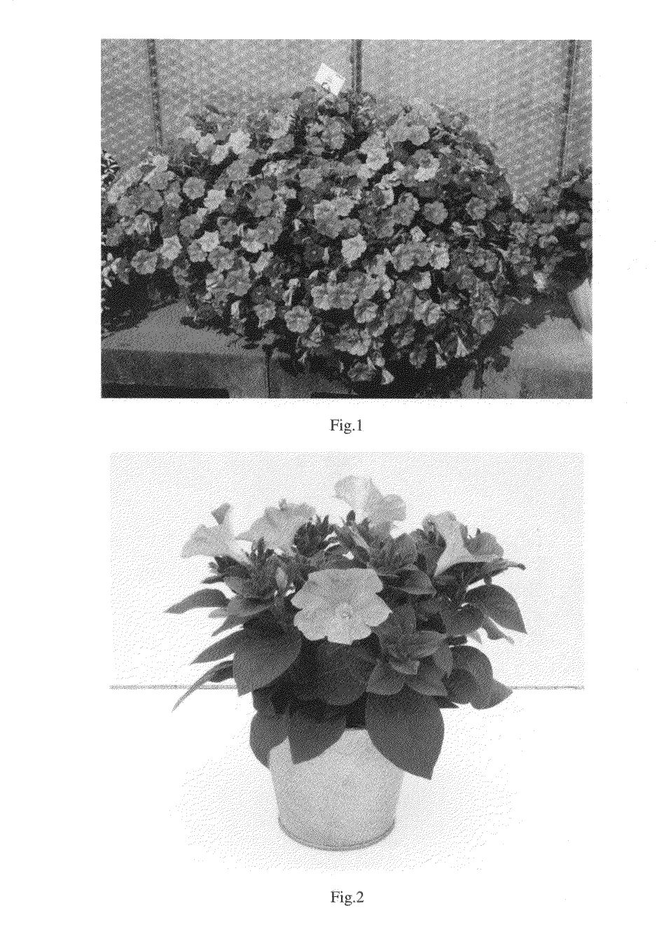

### DESCRIPTION OF THE NEW VARIETY

The following detailed descriptions set forth the distinctive characteristics of ‘KLEPH13228’. The data which define these characteristics were collected from asexual reproductions carried out in Latina, Italy. Data was collected on thirteen-week-old plants grown indoors in Latina, Italy under greenhouse conditions in March of 2014. Color references are to The R.H.S. Colour Chart of The Royal Horticultural Society of London (R.H.S.), 5^{th} edition (2007).
Classification:
   Family.—Solanaceae.
   Species.—Petunia hybrida.
   Common name.—Petunia.
   Variety.—‘KLEPH13228’.
Parentage:
   Female.—Proprietary petunia line ‘PH-2008-0221’.
   Male.—Proprietary petunia line ‘PH-2008-0448’.
Plant:
   Form.—Medium, compact.
   Habit.—Upright, mounding.
   Height (from top of soil).—About 12.0 cm.
   Width (horizontal plant diameter).—About 20.0 cm.
   Propagation.—Terminal tip cuttings and tissue culture.
   Time to produce a finished flowering plant.—13 to 14 weeks in winter, 7 weeks in spring.
   Time to initiate and develop roots.—2 to 3 weeks.
   Root description.—Moderate density, moderate branching, white roots.
Stems:
   Average number (basal).—About 15.0 cm.
   Length of basal branches (from the base of the stem to the tip).—About 18.0 cm.
   Internode length.—0.5 cm to 2.0 cm.
   Diameter of branches (from midpoint).—0.4 cm.
   Stem color.—RHS 143C.
   Anthocyanin.—Absent.
   Texture.—Pubescent.
Leaves:
   Arrangement.—Alternate at the base of the stems, opposite on the flower stems.
   Length.—3.5 cm.
   Width.—2.7 cm.
   Shape.—Obovate.
   Apex.—Broadly acuminate.
   Base.—Attenuate.
   Margin.—Entire.
   Immature leaf color.—Upper surface: RHS N137C Lower surface: RHS 138A.
   Mature leaf color.—Upper surface: RHS N137C Lower surface: RHS 138A.
   Texture.—Both upper and lower surfaces: Slightly pubescent.
   Venation pattern.—Arcuate.
   Venation color.—Both upper and lower surfaces: RHS N144A.
   Petioles.—Length: 1.4 cm Diameter: 0.4 cm Color: RHS 145B Texture: Slightly pubescent.
Flower bud:
   Shape.—Irregular oblong.
   Length.—About 4.0 cm.
   Diameter.—0.6 cm.
   Color at tight bud.—RHS 92D to RHS 145A and RHS 145C.
   Texture.—Slightly pubescent.
Inflorescence:
   Blooming habit (flowering season).—Permanent flowering during summer time.
   Inflorescence type.—Cymose.
   Number of flowers per node.—One.
   Lastingness of individual blooms on the plant.—About one week.
   Fragrance.—Absent.
   Flowers.—Arrangement: Composed of 5 petals fused at the base Shape: Funnel form Diameter (flower face): 5.5 cm Depth (total length of flower): 4.5 cm to 5.0 cm Throat/Funnel: Length: 2.5 cm Diameter (at opening): 1.5 cm Texture: Both inner and outer surfaces: Smooth, glabrous Color, immature flower: Face/Margin: RHS N87B Upper surface: RHS N87B Lower surface: RHS N87B Color, mature flower: Face/Margin color: RHS N82B Upper surface: RHS N82C Lower surface: RHS N82D.
   Petals.—Color: Inner surface: RHS N82B Outer surface: RHS N82D Apex: Mucronate Base: Fused Margin: Entire Strength of waviness: Weak Degree of lobation: Medium to weak.
   Calyx arrangement.—Actinomorphic, composed of 5 sepals.
   Sepals.—Color: Upper surface: RHS N137A Lower surface: RHS 137C Length: 2.0 cm Width: 0.6 cm Shape: Narrowly oblong Apex: Acute Base: Attenuate Margin: Entire Texture: Both upper and lower surfaces: Slightly pubescent.
   Pedicels.—Color: RHS 144A Length: About 3.0 cm Diameter: 0.2 cm Texture: Pubescent.
Reproductive organs:
   Stamens.—Quantity: 5 Shape: Needle with elliptic head Filament: Length: 1.8 cm Diameter: 0.05 cm Color: RHS 145D Anther: Shape: Ovate Color: RHS 4D Length: 0.25 cm Diameter: 0.2 cm Pollen: Color: RHS 4D Amount: Moderate.
   Pistils.—Quantity per flower: 1 Length: 2.2 cm Stigma color: RHS N138C Style color: RHS 145B.
   Fruit and seed set.—No fruit and seed set observed.
   Disease and pest resistance.—No disease and pest/insect resistance observed.

### COMPARISON WITH PARENTAL LINES AND KNOWN VARIETY

‘KLEPH13228’ is a distinct variety of petunia. ‘KLEPH13228’ is distinguished from its parents as described in Table 1:

TABLE 1
Comparison with Parental Lines

| | Variety | Female parent | Male Parent |
|---|---|---|---|
| Trait | ‘KLEPH13228’ | ‘PH-2008-0221’ | ‘PH-2008-0488’ |
| Flower type | Single | Double | |
| Flower color | Light blue | Salmon | Blue |

‘KLEPH13228’ is most similar to the commercial petunia plant named ‘AlpeTunia Light Blue’ (not patented). Differences between the two varieties are described in Table 2:

TABLE 2
Comparison with Similar Variety

| | | Commercial line |
|---|---|---|
| | Variety | ‘AlpeTunia |
| Trait | ‘KLEPH13228’ | Light Blue’ |
| Plant habit | Mounding, | Hanging and |
| | medium-compact | vigorous |
| | vigour | |

We claim:

1. A new and distinct variety of petunia plant named ‘KLEPH13228’ as shown and described herein.
